(19) United States
(12) Patent Application Publication
(10) Pub. No.: US 2015/0141874 A1
(43) Pub. Date: May 21, 2015

(54) Multi-beam Ultrasound Device

(71) Applicant: Kitchener Clark Wilson, Santa Barbara, CA (US)

(72) Inventor: Kitchener Clark Wilson, Santa Barbara, CA (US)

(21) Appl. No.: 14/082,541

(22) Filed: Nov. 18, 2013

(51) Int. Cl. A61N 7/02; A61B 5/00; A61B 5/01

(52) U.S. Cl. CPC A61N 7/02; A61B 5/01; A61B 5/4836; A61N 2007/0078
USPC 601/3

(57) ABSTRACT

A multi-beam ultrasound device with intersecting beams is presented for use in medical therapy and industry. Multiple ultrasound beams interfere constructively and destructively depending on their phase at the target tissue, and this device and its methodology uses phase control for intensity enhancement at the beam intersection. Two among many embodiments are detailed: phase matching where the phases of the beams are determined and controlled to be equal, and phase sweeping where the phases of the beams are purposely varied.

### RELATED APPLICATIONS

This application claims the benefit of U.S. Provisional Application Ser. No. 61/725,351 filed 12 Nov. 2012.

### BACKGROUND OF THE INVENTION

1. Field of the Invention

The invention relates generally to devices for applying ultrasound directed energy. More particularly, the present invention relates to apparatus and methods to apply ultrasound for medical therapy.

2. General Background and State of the Art

Surgical intervention has long been the standard of care to correct physical tissue defects including tumors, atrial fibrillation, and arteriosclerosis. Minimally invasive endoscopic or laparoscopic surgery has been introduced to minimize trauma and speed recovery. In an effort to avoid surgery entirely and to provide care where surgery is impossible, directed ionizing radiation beams, such as X-ray, have been developed to affect tissue remotely by damaging the DNA and causing tissue death by halting the production of proteins required for life.

Ultrasound is another form of directed energy that avoids the significant side affects and risks of ionizing radiation. Ultrasound affects tissue by propagating an acoustic pressure wave, and this wave causes slight vibration of the tissue in its path. The tissue resistance to vibration generates heat, and the sufficiently prolonged application of heat causes temperature rise and cellular death by denaturizing proteins and enzymes required for life, and by cavitation. Denaturation is where the shape of a biological molecule is altered such that it loses its biologic function, and cavitation is the creation of gas bubbles that implode and cause general tissue damage. As in the case of ionizing energy beams, efforts must be made to avoid damage to healthy tissue surrounding the target tissue.

High Intensity Focused Ultrasound (HIFU) is the current state of the art. HIFU emits a focused acoustic beam that, initially wide, focuses down to a small volume at the target tissue. Away from the focus the beam intensity is low so as not to damage healthy tissue while the intensity at the ellipsoidal (1 mm by 10 mm-5 mm³) focus does damage. The small incremental ellipsoid is scanned through the target tissue by moving the beam. HIFU is used in conjunction with a Magnetic Resonance Imaging (MRI), an imaging device that also senses heat, to verify the location of the focus before full power is applied. Even with MRI, the Federal Drug Administration (FDA) only sanctions use of HIFU for uterine fibroid tumors where there is low risk of scattering the high intensity onto healthy tissue by bone and gas pockets. To minimize inadvertent scattering, the patient is fully anesthetized during the 3-5 hour fibroid procedure while prone within the MRI.

There is a need to have a means to introduce surgically effective ultrasound that is safer than HIFU, can be applied more widely than to uterine fibroids, and does not require an expensive MRI watchdog device. It is further advantageous to treat an incremental volume greater than the 5 mm³ HIFU focus.

### BRIEF SUMMARY OF THE INVENTION

The fundamental concept behind this invention is this use of at least two ultrasound transducers with each having an acoustic beam with intensity too low to affect tissue, but arranged to intersect at the target volume with controlled relative phase. The controlled phase adjusts the beam interference such that the resultant is a merged beam of greater intensity, no longer benign, that can affect the targeted tissue and not surrounding tissue.

The object of the invention is to provide a therapeutically effective ultrasound device that can go where HIFU cannot, is safer, is less expensive, does not require an MRI, and handles larger incremental volumes. Further objects and advantages of the present invention will become more apparent from the following description of the preferred embodiments which, taken in conjunction with the accompanying drawings, illustrate, by way of example, the principles of the invention.

### DESCRIPTION OF THE DRAWINGS

FIG. 1 is a graphical depiction of the approximate time cellular temperature must be held for cellular death by denaturation.

FIG. 2 is a schematic representation of existing art HIFU therapeutic ultrasound.

FIG. 3 is a schematic representation of (a) an ultrasound beam from an unfocused transducer and (b-d) the three ways two such beams can intersect.

FIG. 4 is a graphical display of measurements of the power delivered by a single 1 MHz 19 mm unfocused ultrasound transducer showing the contours of the −3 db, −6 db and −9 db peak power regions.

FIG. 5 is a graphical representation of the calculated intensity delivered by a single 19 mm unfocused transducer in soft tissue operated at 0.25, 0.50, 1.0, 1.5, and 2.0 MHz and illustrating the effect of frequency and distance on attenuation and beam divergence.

FIG. 6 is a graphical representation of the calculated and measured power gain, as a function of relative phase at the drivers, of a point within the intersection of two unequal 40 MHz transducers in water, and the projected power gain had they been equal.

FIG. 7 (a) is a graphical display of power from measurements of two 30° intersecting ultrasound beams from two equal 1 MHz 19 mm unfocused transducers with fixed phase and showing the contours of the 90%, −3 db, −6 db and −9 db peak power regions; (b) is a graphical display of power from measurements of two 60° intersecting ultrasound beams from two equal 1 MHz 19 mm unfocused transducers with fixed phase and showing the contours of the 90%, −3 db, −6 db and −9 db peak power regions.

FIG. 8 is a graphical display of power from measurements of two 30° intersecting ultrasound beams from two equal 1 MHz 19 mm unfocused transducers with swept phase and showing the contours of the 90%, −3 db (50%), −6 db (25%) and −9 db (12.5%) peak power regions.

FIG. 9 is a schematic representation of intersecting transducers using match phase approach.

FIG. 10 is a schematic representation of a phase matching drive generator.

FIG. 11 is a schematic representation of a phase match and shifter having phase estimating capability.

FIG. 12 is a flowchart of an algorithm for controlling phase using the phase match and shifter having phase estimating capability.

FIG. 13 is (a) a schematic representation of an analog embodiment of the phase estimator; (b) a schematic representation of analog embodiment of the estimate phase shifter.

FIG. 14 is a schematic representation of a phase match and shifter having power maximizing capability.

FIG. 15 is a flowchart of an algorithm to estimate phase by maximizing power.

FIG. 16 is a schematic representation of intersecting transducers using swept phase approach.

FIG. 17 is a schematic representation of a phase sweeping drive generator using two ultrasound oscillators.

FIG. 18 is a schematic representation of a phase sweeping drive generator using a single ultrasound oscillator.

FIG. 19 is a schematic representation of a phase sweeping phase shifter.

FIG. 20 is a schematic representing a swept phase generator using a low frequency oscillator.

### EXISTING ART

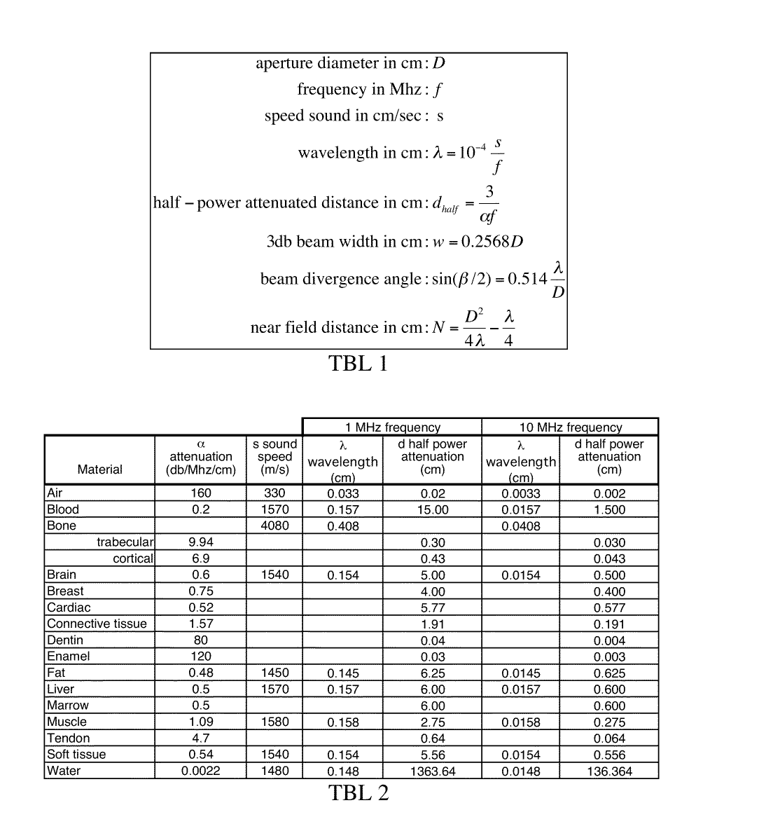

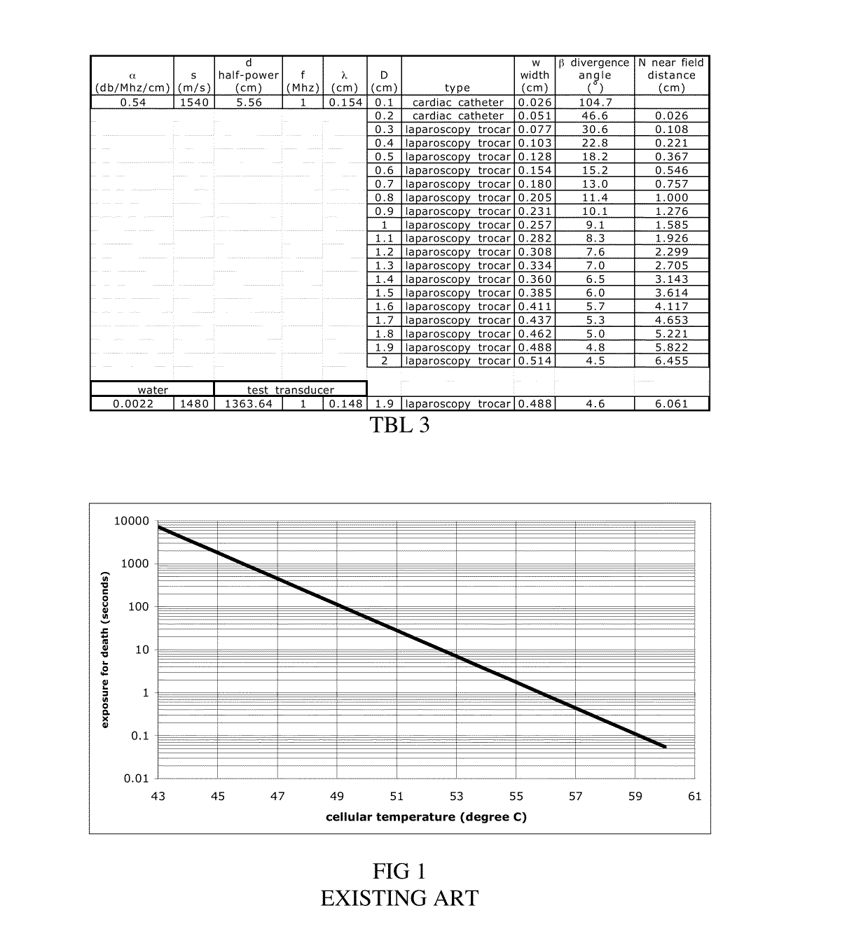

The common purpose of ultrasound application is to warm the tissue sufficiently and for a sufficient time to cause death. As a rule-of-thumb, this combination of tissue temperature and application time begins with the minimal combination of 43° C. for 120 minutes, with the duration falling by half with each 1° C. rise thereafter as illustrated in FIG. 1. Another purpose would be to trigger cavitation implosions, but these are difficult to control and less commonly used.

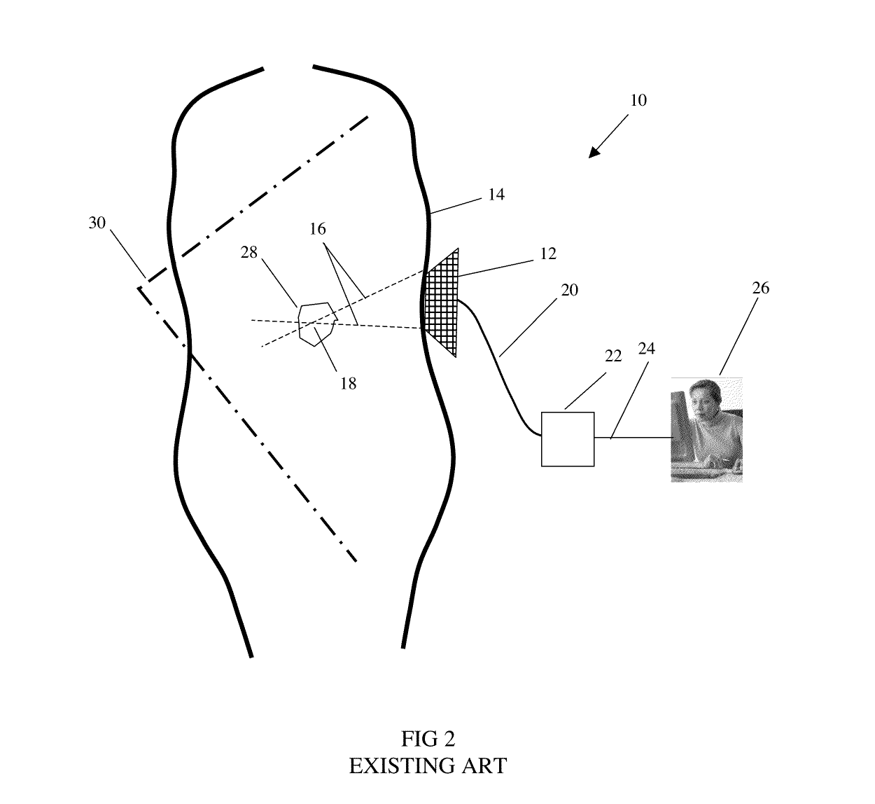

FIG. 2 illustrates a conventional HIFU surgical ultrasound device 10 having an ultrasound HIFU transducer 12 mounted external to the patient surface 14 (typically skin). The transducer emits a conical ultrasound beam 16 with a high intensity focal point 18, and is coupled through an electrical cable 20 to the drive generator and beam direction electronics 22. An operator-computer combination 26, connected to the signal generator and beam direction electronics by a cable 24, controls the operation of the device in order to scan the target volume 28 in small incremental volumes. The manner in which the beam is generated includes having a phased array transducer where the beam shape and direction is selected by judicious phased activation of many small transducers, or by having a single transducer shaped as a portion of a sphere with direction selected by mechanical actuators.

In operation, the operator-computer 26 directs the HIFU beam 16 to form a focus 18 within the target volume 28 at low power and at a desired spot, and an MRI device 30 is operated to scan the patient and image the spot warmed by the low power beam to verify the actual anatomical location of the spot. If the operator-computer determines the focus is located as desired, the HIFU transducer 12 is operated at high power to damage the tissue. This procedure is repeated many times to cover the target volume in small ellipsoidal volume increments of approximately 5 mm³.

### Technical Background and Experimental Results

### General Concept

Ultrasound beams are traveling waves of acoustic pressure produced by ultrasonic transducers. As waves, they interact with other acoustic waves with classical interference patterns having repeating constructive and destructive interference. Destructive interference occurs when traveling waves are out of phase and partially or fully cancel each other, and constructive interference is when the waves are in phase and build on each other. It is possible, using phase control, to modify the interference pattern and apply acoustic beams with low intensities that do not affect surrounding tissue yet have greater therapeutic intensity at their intersection. The invention is to emit a plurality of intersecting ultrasound beams while controlling their phasing such that the resulting intersection at the target tissue takes advantage of interference.

A Single Beam:

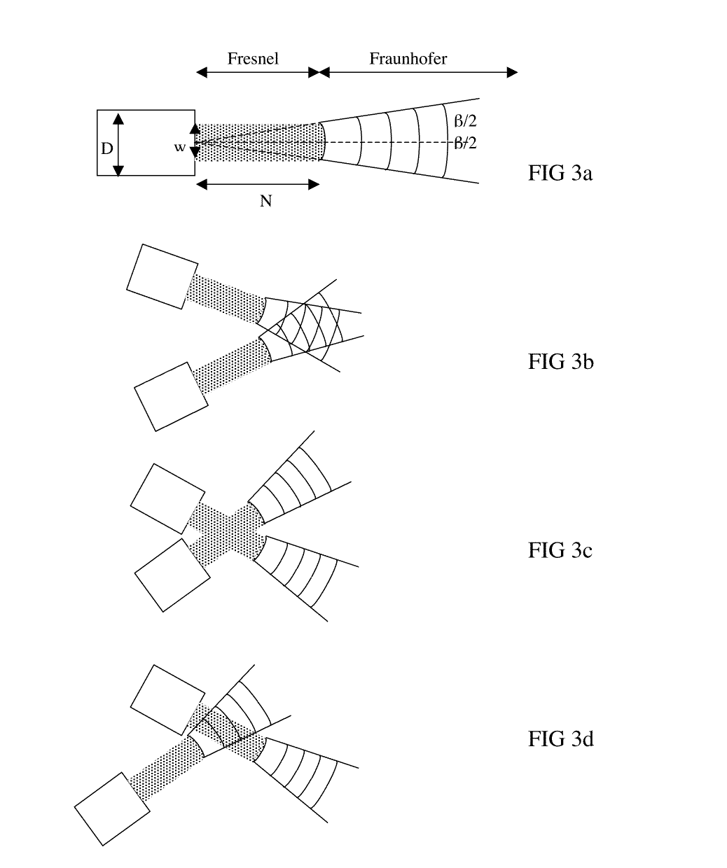

FIG. 3a illustrates a single unfocused transducer having aperture diameter D and designed to generate an unfocused acoustic beam of frequency f into a medium having speed of sound s and acoustic signal attenuation α. As illustrated in FIG. 3a and defined in TBL 1, an unfocused circular transducer produces an acoustic beam having initial nominal width w that diverges with angle β resulting in its energy being dispersed over an increasingly wider area. The beam is stable but poorly defined until the Far Field Fraunhofer region beyond the Near Field Fresnel Zone. Within the Near Field Fresnel zone the beam is essentially cylindrical and interferes with itself as it radiates from multiple points on the vibrating transducer active surface, and the region has multiple interference maxima and minima. Within the Far Field Fraunhofer zone the beam ceases to interfere with itself and becomes an expanding (as per the divergence angle) and well-defined pressure wave. The Near Field distance N is that of the last Fresnel interference maxima and defines the transition from Fresnel to Fraunhofer zones.

TBL 2 presents s and a for various tissues along with, at two frequencies, the calculated wavelength λ and distance to half-power (−3 db) due to attenuation by tissue and scattering. Using soft tissue as the typical target, 1 MHz and 10 MHz beams have, respectively, λ of 0.154 cm and 0.015 cm and have lost half their power after 5.6 cm and 0.56 cm. The frequency of therapeutic ultrasound is typically chosen around 1 MHz to reduce attenuation.

TBL 3 illustrates the effect of various aperture diameters on width w, Near Field distance N and divergence angle β of a 1 MHz unfocused transducer in soft tissue. The tubular enclosure column shows the catheter type or laparoscopy trocar having the same diameter as the transducer.

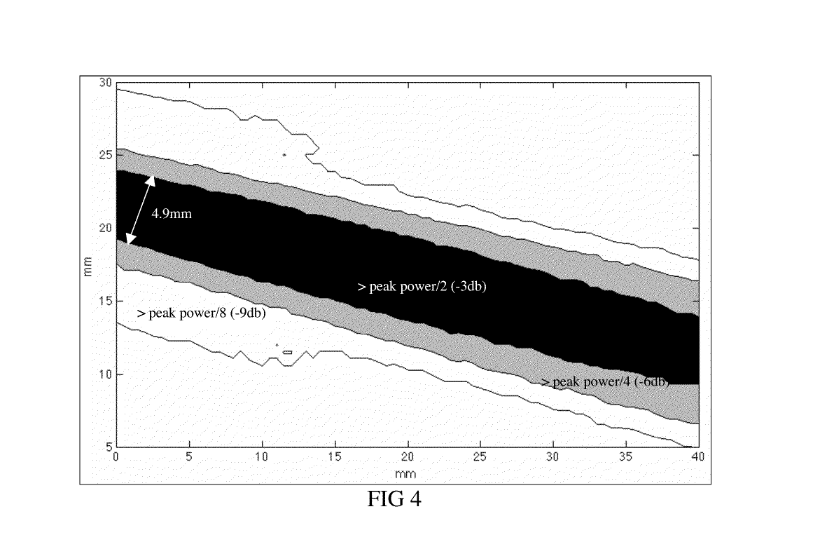

FIG. 4 is a two-dimensional contour plot of the measured peak power along the main axis of a 1 MHz 19 mm diameter unfocused transducer (Olympus A314S-SU) operating in a water bath. Its characteristics are shown in TBL 3 in agreement with FIG. 4.

Therapeutically, the power intensity I (watts/cm²) is the power density applied to tissues. The beam radius along the axis is, essentially, constant in the Fresnel zone but increases in the Fraunhofer zone as the beam diverges after the near field distance N. Tissue attenuation of the signal occurs throughout its path, and the beam intensity is modeled as

$$attenuation \text{:} P = {P}_{0} {10}^{- f α x / 10}$$

$$beam radius \text{:} r = \{ \begin{matrix}{r}_{0} & for x < N \\ {r}_{0} + ( x - N ) tan ( β / 2 ) & for x \geq N\end{matrix} \\ beam area \text{:} A = π {r}^{2} \\ beam intensity \text{:} I = \frac{P}{A}$$

where P₀ is the transducer power output, r₀ is the radius (half-width) of the beam at the aperture, and x is the distance from the aperture. Including the equations of TBL 1, FIG. 5 illustrates the intensity of a 125 mW 19 mm unfocused transducer in soft tissue. Initial Fresnel zone interaction is only attenuation, and the following Fraunhofer zone interactions are attenuation and dispersion due to angular divergence angle β. The lower the transducer frequency, the shorter the Fresnel zone, the greater the dispersion, and the shallower the therapeutic affect.

Scattering:

One challenge ultrasound has is the direction change due to scattering by non-homogenous tissues. Scattering is a general term describing the change of the sound direction of travel due to interaction with non-homogenous tissues, and is a component of acoustic attenuation by tissue. At the boundary between different tissues, having different acoustic impedance and speed of sound, the wavelike properties of sound cause transmission and reflection. If γ₀ is the angle between an incident beam and the vertical to the boundary and s₀ is the incident speed of sound, Snell's Law defines the corresponding angles of the reflected and transmitted portions according to

$${γ}_{reflected} = {γ}_{0}$$

$${γ}_{transmitted} = {sin}^{- 1} ( \frac{{s}_{1}}{{s}_{0}} sin {γ}_{0} )$$

The angle of the transmitted wave is not the same as the incident meaning the transmitted wave is moving in a different direction. The quantity reflected is dependent on the acoustic impedances of the tissue on each side of the boundary with an Intensity Reflection Coefficient given by

$$I R C = \frac{reflected intensity ( W \text{/} {cm}^{2} )}{incident intensity ( W \text{/} {cm}^{2} )} = {( \frac{{Z}_{1} - {Z}_{0}}{{Z}_{1} + {Z}_{0}} )}^{2}$$

where Z₀ is the acoustic impedance of the source side of the boundary, and Z₁ is that of the transmitted side. Air (contents of the intestine, stomach, lungs) and bone have dramatically different impedance than tissue and will strongly reflect sound.

Intersection of Two Beams:

FIGS. 3b, 3c and 3d illustrate three types of intersections: FIG. 3b Fraunhofer-Fraunhofer, FIG. 3c Fresnel-Fresnel, and FIG. 3d Fresnel-Fraunhofer. All three types form standing interference patterns given continuous sinusoidal excitation. Given a relative phase φ, at a point in the intersection, a sinusoidal pressure wave from a first transducer beam a_{first} sin(ωt) and a sinusoidal pressure wave from a second transducer beam a_{second} sin(ωt+φ) combine by superposition and sum to form a resultant sinusoidal pressure wave with amplitude given by the well known sum-of-sines-with-equal-frequency equation

aᵣₑₛᵤₗₜₐₙₜ=\sqrt{a²_{first}+a²_{second}+2a_{first}a_{second} cos φ}

The peak power in the resultant is proportional to the square of the amplitude:

Pᵣₑₛᵤₗₜₐₙₜ∝a²_{first}+a²_{second}+2a_{first}a_{second} cos φ (1)

Compared to the peak power of the first transducer, the peak power gain is

$$\begin{matrix}{G}_{P} = \frac{{a}_{first}^{2} + {a}_{second}^{2} + 2 {a}_{first} {a}_{second} cos φ}{{a}_{first}^{2}} \\ = 1 + \frac{{a}_{second}^{2} + 2 {a}_{first} {a}_{second} cos φ}{{a}_{first}^{2}} \\ = 1 + \frac{{a}_{second}^{2}}{{a}_{first}^{2}} + \frac{2 {a}_{second}}{{a}_{first}} cos φ\end{matrix} \qquad ( 2 )$$

If a_{first}=a_{second} then

G_{P}=2(1+cos φ)

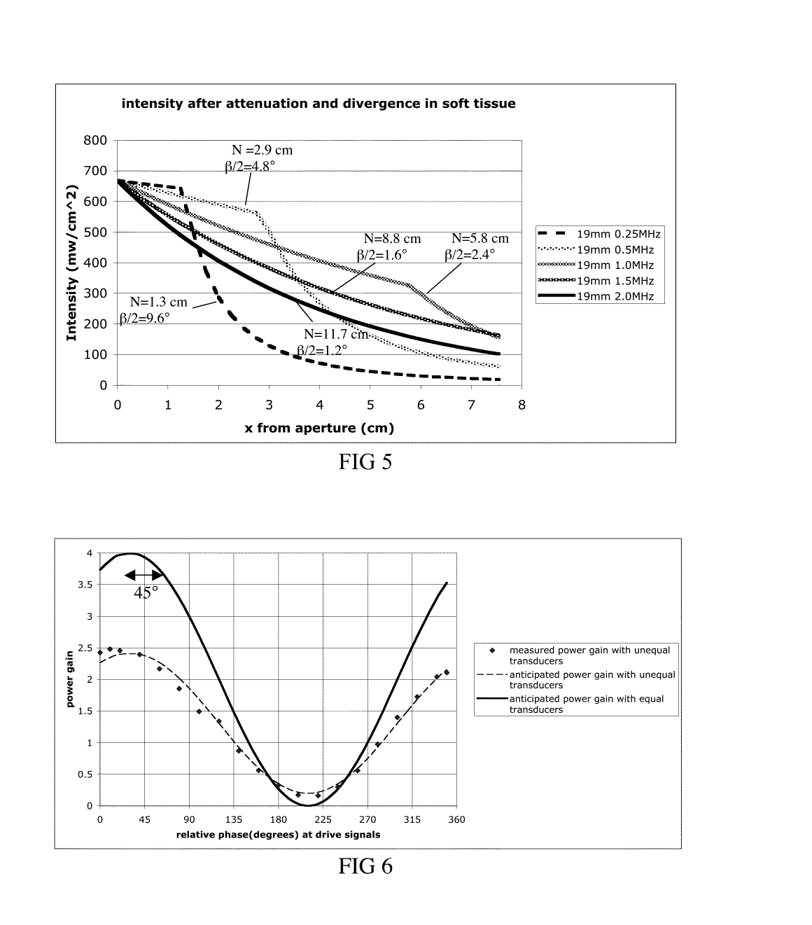

and the power gain is maximum 4× when φ=0 and minimum 0× when φ=π. FIG. 6 shows tight agreement between the measured and expected power gain G_{P}, from Eq. 2, for two intersecting 40 MHz transducers in water with various phase values, and measured by a fixed hydrophone. The hydrophone measured the signals generated by the two transducers, one at a time, as having amplitudes: a_{first}=8.56 mV and a_{second}=4.74 mV. The phase values are set at the transducer drive sources, and not at the intersection where the hydrophone was fixed, and thus have a 35° offset due to electronics and relative transducer positions from the hydrophone. Also shown is the equivalent power curve should a_{first}=a_{second} illustrating the anticipated 0× min and 4× max values.

FIG. 6 shows the maximum power point is not greatly sensitive to phase: 90% of the peak power is contained within ±45° of the optimum phase.

Fixed Phase Intersections:

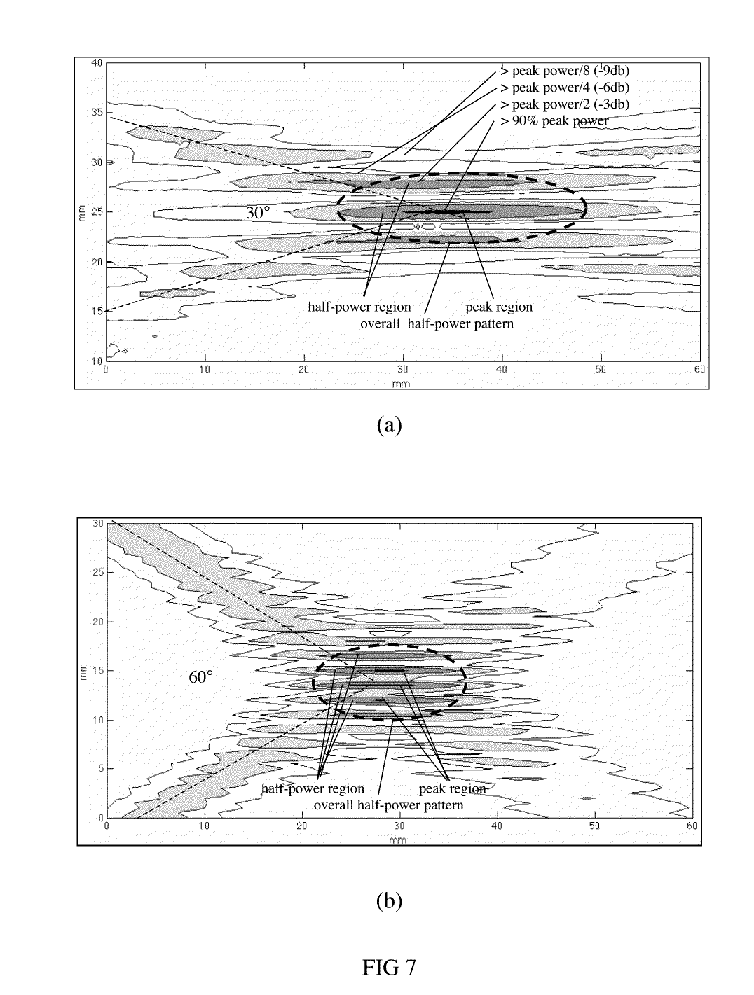

FIG. 7a presents the measured power contours from two Fraunhofer-Fraunhofer intersecting beams (FIG. 3b) in water from identical 1 MHz 19 mm transducers of FIG. 4 with a 30° angle between them. The transducers have had their drive signals phased so as to produce a fixed matched-phase peak at the center of image. Several islands of peaks and valleys are seen, and the small (less than 1 mm×10 mm) black 90% peak power region has power gain 4× that of the arriving beams (light gray). The dual 4.8 mm wide beams produce several half-power regions (dark gray), and an ellipsoidal overall half-power pattern that is around 7 mm×25 mm (641 mm³).

FIG. 7b shows the measured power contours of 60° angular intersection in water and having a larger number of islands of peaks and valleys with a shorter ellipsoidal overall half-power pattern of around 7 mm×17 mm. As the intersection angle approaches 90°, the overall pattern is expected to approach spherical with nearly a 7 mm diameter (179 mm³).

Swept Phase Intersections:

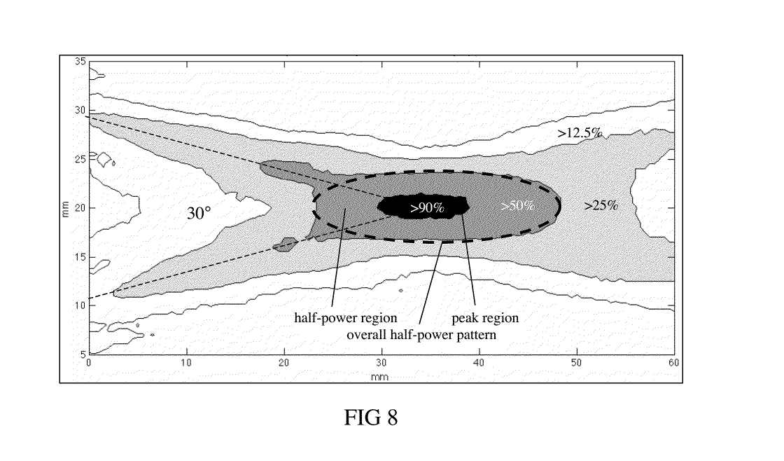

Changing the relative phase of the intersecting beams moves the interference patterns but not the overall half-power pattern. FIG. 8 presents the measured power contours from two Fraunhofer-Fraunhofer intersecting beams in water from identical 1 MHz 19 mm transducers of FIG. 4 having a 30° angle between them, and where the transducers have their relative phase of their drive signals continuously and linearly swept from 0° to 360°. The islands of peaks and valleys are eliminated, and the peak region is 2.5 mm×10 mm and remains 4× that of the arriving beams. The 4.8 mm beams produce an overall half-power pattern that is 7 mm×25 mm, the same as for FIG. 6 of the fixed phase intersection.

Linear sweeping of phase means φ(t)=φt where

$$\dot{φ} = \frac{d }{d t} φ ( t )$$

is the phase sweep rate. The swept drive signal is then

sin [ωt+φ(t)]=sin [ωt+φt] (3)

sin [ωt+φ(t)]=sin [ω+φt] (4)

This establishes that a swept phase angle is produced by drive signals with differing frequencies where the difference is the phase sweep rate.

### DETAILED DESCRIPTION OF PREFERRED EMBODIMENT

Based on the experimental and theoretical considerations presented in the Technical Background and Experimental Results section, two among many approaches to operate intersecting ultrasound transducers are here described. Both approaches produce a 4× power gain at the beam intersection compared to surrounding tissue.

The first approach is a matched phase approach where the beams at the intersection are made to have essentially equal phase to take advantage of constructive interference, and this approach is shown to have at least two implementations: one that estimates the individual transducer phases at the target site and directly constructs a phase match, and a second that maximizes the measured power delivered to the target site and indirectly constructs a phase match.

The second approach to operate intersecting ultrasound transducers is a swept phase implementation that smoothes the interference peaks and valleys and mitigates the wave cancellation interference pattern.

### Intersecting Transducers Using Matched Phase Approach

Ultrasound energy does not have a particle equivalence; it has only wave properties, and waves interact with each other, canceling and summing depending on their relative phase locally at the target. Wave summation is taken advantage of by using intersecting ultrasound beams having identical frequency and phase matched at the target tissue, as in FIG. 7a-b. Small (1 mm×10 mm, similar to HIFU) high intensity areas (>90%) are produced with a power gain of 4× relative to tissue in the non-intersecting regions. Fraunhofer-Fraunhofer intersections are preferred where the intersecting beams are well defined, as in FIG. 3b. This implementation requires an ultrasonic sensor at or near the intersection of the transducer beams to report the transducer ultrasound signals, and the sensor may be configured to include a temperature probe to monitor therapy as per FIG. 1.

The locations of the transducers and sensor are arranged directly by the medical professional performing the ultrasound therapy using manual, minimally invasive, or other means. Unlike HIFU, the transducers are very close, or proximal, to the target tissue.

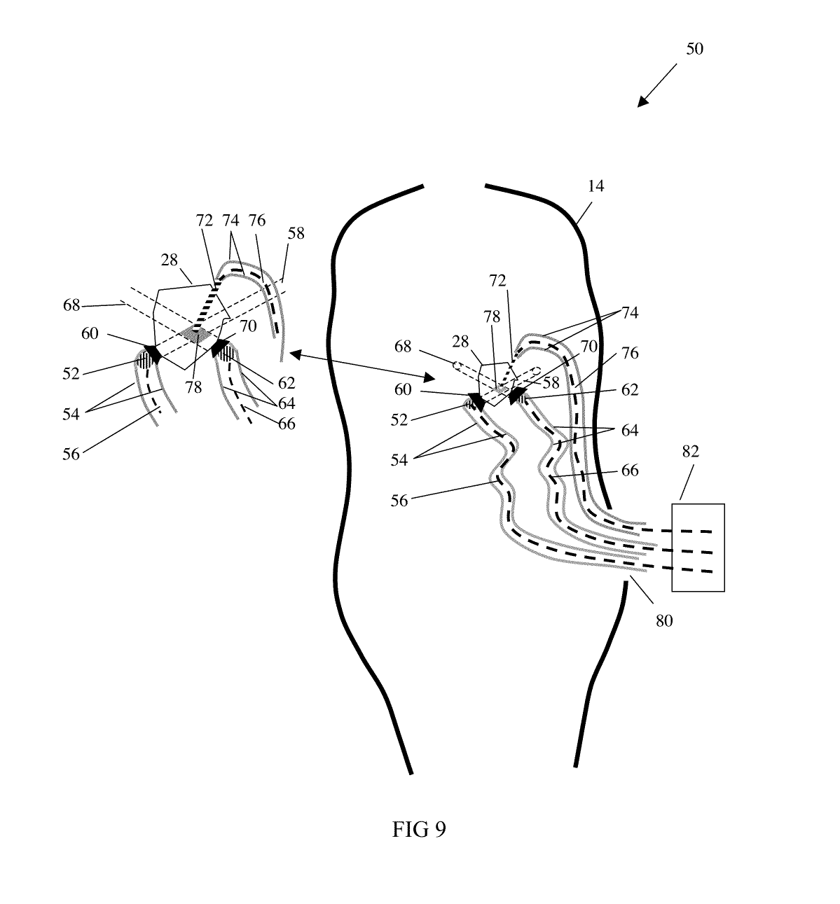

FIG. 9 illustrates the intersecting transducers using matched phase approach 50. A reference ultrasound transducer 52, a slave ultrasound transducer 62, and a sensor 72 responsive to ultrasonic pressure, and optionally including temperature sensing capabilities, are introduced through the patient surface 14 using minimally invasive surgical methods through portal 80 in order to treat an internal target tissue volume 28. The sensor is preferably placed within the intersection 78, and the reference transducer beam 58 is made to intersect with the slave transducer beam 68 in preferably a Fraunhofer-Fraunhofer manner. If the minimally invasive technique includes CO₂ to expand the region for surgical access, as in laparoscopy, the transducers include a reference couplant 60 and a slave couplant 70 to impedance match and route the ultrasound to the tissue through the gas. As needed, the reference transducer, slave transducer, and sensor are introduced through a reference tube 54, a slave tube 64, and a sensor tube 74 which may be distinct or combined. The transducers and sensor are connected to a phase matching drive generator 82 using reference cable 56, slave cable 66, and sensor cable 76.

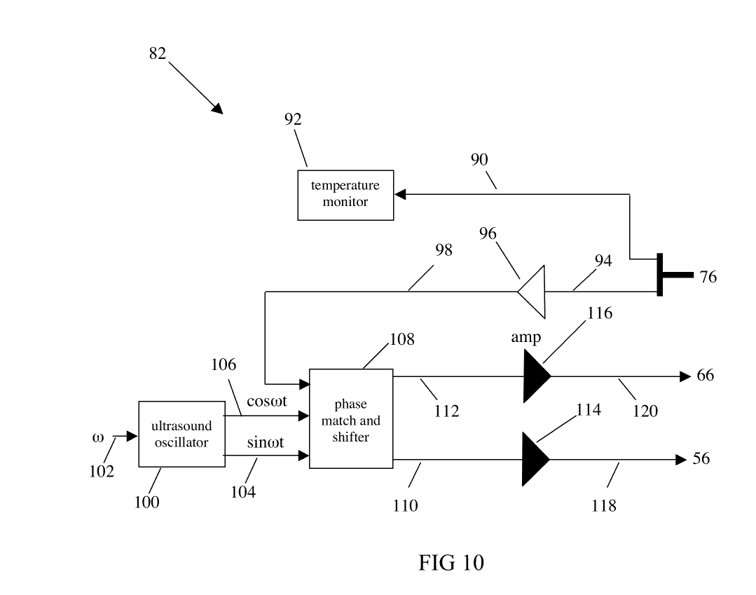

As illustrated in FIG. 10 of the phase matching drive generator 82, the sensor 72 temperature signal 90, if any, is delivered over sensor cable 76 and is used to monitor 92 the progress of therapy. The sensor ultrasound signal 94 is buffered by preamp 96 resulting in the buffered ultrasound signal 98. An ultrasound oscillator 100 using frequency 102 produces sine 104 and cosine 106 (sine signal 104 phase shifted by 90°) signals that, along with the buffered ultrasound signal, are used by the phase match and shifter 108 to generate a precursor reference drive signal 110 and a precursor slave drive signal 112. The precursor drive signals are amplified and impedance matched by amplifiers 114 and 116 to become the reference drive signal 118 and slave drive signal 120 that are routed to the reference cable 56 and the slave cable 66. Preferably, a single ultrasound oscillator is used to produce the sine and cosine signals to stabilize phase and minimize phase sweeping, as per Eq. 4, to avoid oscillator drift and frequency tolerances.

Phase Match and Shifter 108 Implemented Using Phase Estimation:

The reference transducer 52 drive signal 118 is described as

ω=2πf

drive_{reference}(t)=a_{reference} sin ωt

and the slave transducer 62 φₛₗₐᵥₑ phase shifted drive signal 120 as

driveₛₗₐᵥₑ(t)=aₛₗₐᵥₑ sin(ωt+φₛₗₐᵥₑ)

At the location of the sensor 72, the reference ultrasound beam 58 and slave ultrasound beam 68 are described as

sensor_{reference}(t)=k_{reference}a_{reference} sin(ωt+φ_{reference})

sensorₛₗₐᵥₑ(t)=kₛₗₐᵥₑaₛₗₐᵥₑ sin(ωt+φₛₗₐᵥₑ+φₛₗₐᵥₑ)

where the k's represent the effects of amplifier and transducer gains and signal degradation due to attenuation. The φ's are the additional phases introduced by electronics (e.g. amplifier, cabling, piezo-electric actuator, . . . ), and transducer positioning relative to the sensor. The superposition of these two intersecting transducer ultrasound beams produces a resultant signal described using complex mathematics as

$$j = \sqrt{- 1}$$

$${e}^{j x} = cos x + j sin x$$

$${sensor}_{reference} ( j ω ) = {k}_{reference} {a}_{reference} {e}^{j ( ω t + {ϕ}_{reference} )}$$

$${sensor}_{slave} ( j ω ) = {k}_{slave} {a}_{slave} {e}^{j ( ω t + {φ}_{slave} + {ϕ}_{slave} )}$$

$${sensor}_{resultant} ( j ω ) = {a}_{resultant } {e}^{j ( ω t + {ϕ}_{resultant} )}$$

$${a}_{resultant} {e}^{j ( ω t + {ϕ}_{resultant} )} = {k}_{reference} {a}_{reference} {e}^{j ( ω t + {ϕ}_{reference} )} + {k}_{slave} {a}_{slave} {e}^{j ( ω t + {φ}_{slave} + {ϕ}_{slave} )}$$

with the associated phasor equation

$${a}_{resultant} {e}^{j {ϕ}_{resultant}} = {k}_{reference} {a}_{reference} {e}^{{jϕ}_{reference}} + {k}_{slave} {a}_{slave} {e}^{j ( {φ}_{slave} + {ϕ}_{slave} )}$$

The resultant sinusoid magnitude is calculated as

$$\begin{matrix}{a}_{resultant} = \vert {k}_{reference} {a}_{reference} {e}^{{jϕ}_{reference}} + {k}_{slave} {a}_{slave} {e}^{j ( {φ}_{slave} + {ϕ}_{slave} )} \vert \\ = \sqrt{\begin{matrix}{[ {k}_{reference} {a}_{reference} cos {ϕ}_{reference} + {k}_{slave} {a}_{slave} cos ( {φ}_{slave} + {ϕ}_{slave} ) ]}^{2} + \\ {[ {k}_{reference} {a}_{reference} sin {ϕ}_{reference} + {k}_{slave} {a}_{slave} sin ( {φ}_{slave} + {ϕ}_{slave} ) ]}^{2}\end{matrix}} \\ = \sqrt{\begin{matrix}{( {k}_{reference} {a}_{reference} )}^{2} + {( {k}_{slave} {a}_{slave} )}^{2} + \\ 2 {k}_{reference} {k}_{slave} {a}_{reference} {a}_{slave} cos ( {ϕ}_{reference} - {φ}_{slave} - {ϕ}_{slave} )\end{matrix}}\end{matrix}$$

with proportional power

powerᵣₑₛᵤₗₜₐₙₜ∝(k_{reference}a_{reference})²+(kₛₗₐᵥₑaₛₗₐᵥₑ)²2k_{reference}kₛₗₐᵥₑa_{reference}aₛₗₐᵥₑ cos(φ_{reference}−φₛₗₐᵥₑ−φₛₗₐᵥₑ) (5)

Eq. 5, a generalized version of Eq. 1, establishes that the power of the resultant is maximized when ω_{reference}−φₛₗₐᵥₑ−φₛₗₐᵥₑ=0 or

φₛₗₐᵥₑ=φ_{reference}−φₛₗₐᵥₑ (6)

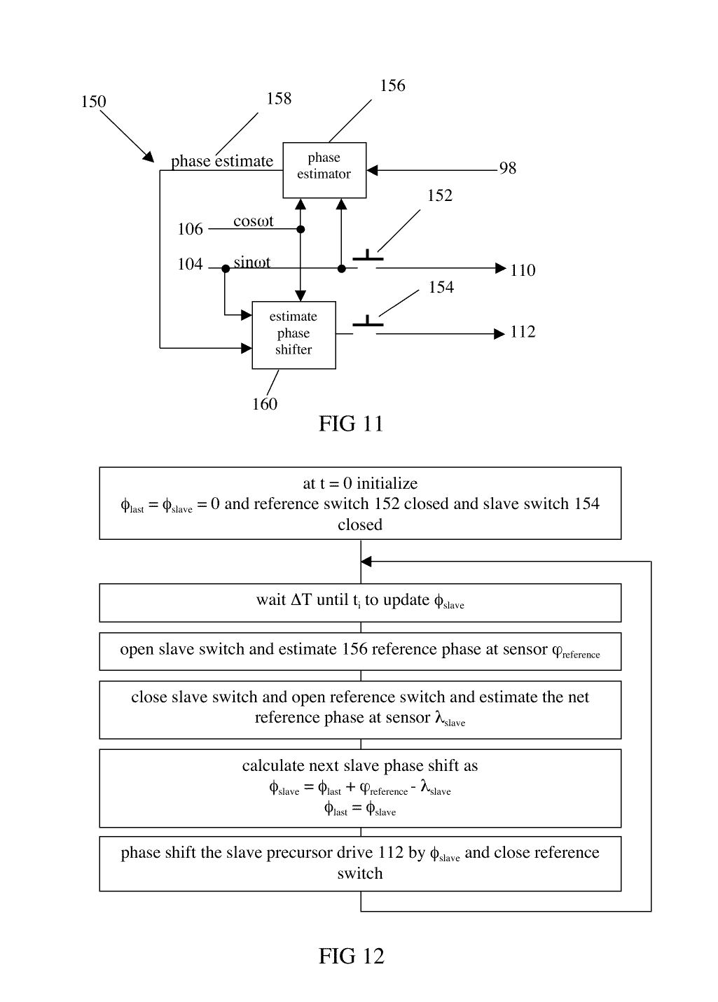

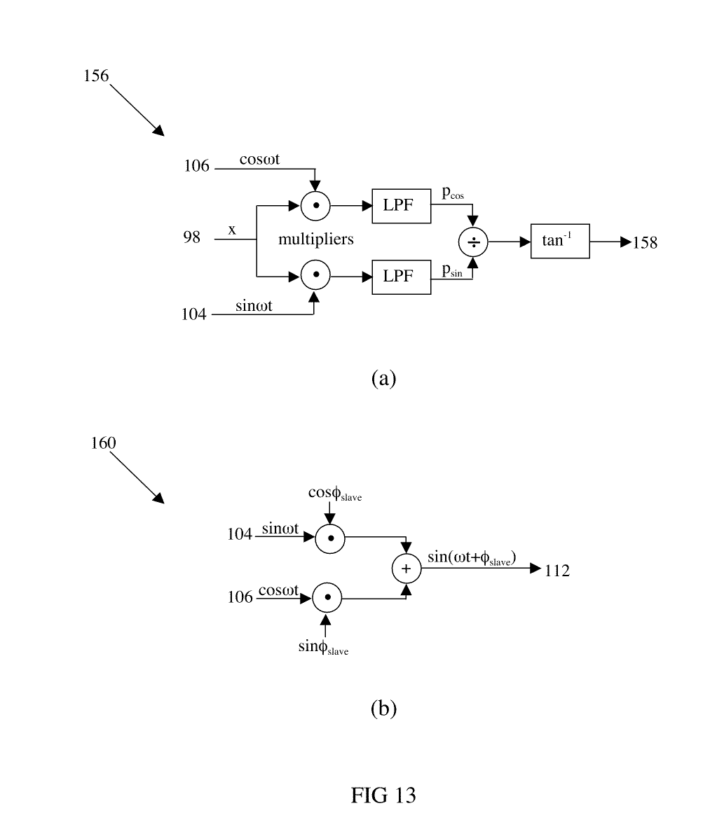

As shown in FIG. 11, one embodiment 150 of the phase match and shifter 108 uses the buffered ultrasound signal 98 magnitude to estimate, individually, the phases of the reference transducer beam 58 and slave transducer beam 68 at the sensor 72. The desired slave phase φₛₗₐᵥₑ applied to the slave precursor drive signal 112 is determined based on Eq. 6. As illustrated in FIG. 6, φₛₗₐᵥₑ does not have to be precisely known and ±45° is sufficient to contain 90% of the maximum power. This phase match and shifter 108 with phase estimation 150 includes two switches 152 and 154 to null the slave precursor drive signal or the reference precursor drive signal 110. A phase estimator 156 responds to the buffered ultrasound signal, and the resulting phase estimate 158 is used by the estimate phase shifter 160 to generate the slave precursor drive signal 112.

With only the reference transducer 52 active (reference switch 152 closed and slave switch 154 open), the sensor 72 ultrasound signal 94 represents only the reference beam 58 ultrasound signal, and the phase estimator 156 phase estimate 158 is that of the reference phase (P_{reference} at the sensor site. With only the slave transducer 62 active (reference switch 152 open and slave switch 154 closed), the phase estimate is that of the net slave phase λₛₗₐᵥₑ at the sensor site including the current known slave phase shift: λₛₗₐᵥₑ=φₛₗₐᵥₑ+φₛₗₐᵥₑ. φₛₗₐᵥₑ is calculated as φₛₗₐᵥₑ=λₛₗₐᵥₑ−φₛₗₐᵥₑ and the next φₛₗₐᵥₑ is calculated as φₛₗₐᵥₑ+φ_{reference}−λₛₗₐᵥₑ.

FIG. 12 presents a basic algorithm for calculating and implementing the slave phase adjustment. The loop timing ΔT is selected to sufficiently sample the beam phases to accommodate the 100 Hz or so transducer movement spectrum. At time zero, φₗₐₛₜ=φₛₗₐᵥₑ=0 and all switches are closed so that the reference and slave drives are normally active. After each period ΔT, the slave switch 154 is opened (no slave drive) and the phase estimator 156 responds to the buffered ultrasound signal 98 to estimate its phase 158 representing φ_{reference} of the reference beam 58. Following, the slave switch is closed, the reference switch 152 opened (no reference drive), and the phase estimator responds to the buffered ultrasound signal to estimate its phase representing λₛₗₐᵥₑ of the slave beam 68. Following, the estimate phase shifter 160 calculates the desired φₛₗₐᵥₑ=φₗₐₛₜ+φ_{reference}−λₛₗₐᵥₑ, φₗₐₛₜ=φₛₗₐᵥₑ, phase shifts the slave precursor drive 112 accordingly, and closes the reference switch for the next cycle.

Analog Phase Estimation: As those skilled in the art can attest, phase estimation 156 is a common activity in engineering and is performed using analog circuitry as follows. Given sin ωt and cos ωt, the products of these with the buffered ultrasound signal 98, represented generally by a sin(ωt+φ), are formed as

$$\begin{matrix}x = a sin ( ω t + φ ) \\ {p}_{sin} = x sin ω t \\ = \frac{1}{2} a cos φ - \frac{1}{2} a cos φcos 2 ω t + \frac{1}{2} a sin φsin 2 ω t\end{matrix}$$

$$\begin{matrix}{p}_{cos} = x cos ω t \\ = \frac{1}{2} a sin φ + \frac{1}{2} a cos φsin 2 ω t + \frac{1}{2} a sin φ cos 2 ω t\end{matrix}$$

These products consist of a dc phase component on which is superimposed ac components having frequency 2ω. Simple low-pass filtering (LPF) of these products at below 2ω produces the sine and cosine components of the phase: ½ a cos φ and ½ a sin φ. The ratio of these values

$$tan φ = \frac{sin φ}{cos φ} = \frac{\frac{1}{2} a sin φ}{\frac{1}{2} a cos φ}$$

is the tangent of the phase angle, and phase is determined using the inverse tangent

$$φ = {tan}^{- 1} ( \frac{LPF ( x cos φ )}{LPF ( x sin φ )} )$$

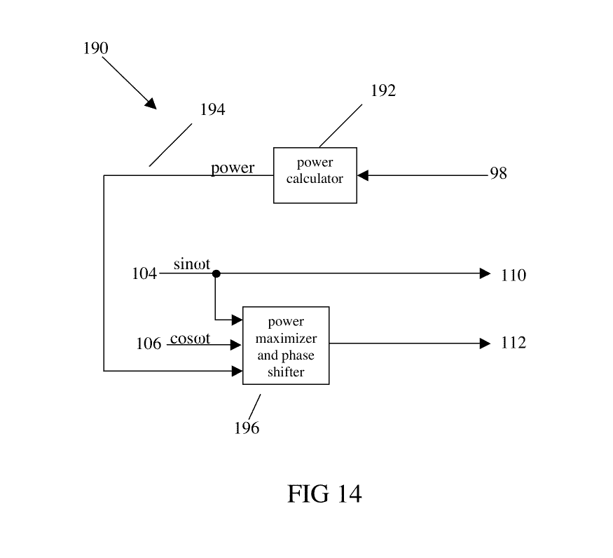

A block diagram of an analog embodiment 170 of the phase estimator 156 is presented in FIG. 14a. Typically therapeutic ultrasound frequencies are in the 0.5-8 MHz range, and the two FIG. 14a multipliers and LPFs are implemented in hardware where the multiplier must handle signals at least twice that frequency (see the 2ω terms in the pₛᵢₙ and p_{cos} equations above). At least a 50 MHz multiplier is preferable, and devices such as the Analog Devices AD835 is a 250 MHz dc-coupled four-quadrant multiplier suitable for this application. The LPF is preferably a simple op-amp or R-C circuit with break frequency set at a few KHz to capture the dc components of the multiplier outputs, and an analog-digital converter takes the LPF outputs into a computer, or other calculating electronic device, for division and arc-tangent operations for phase calculation. The low frequency division and inverse tangent operations can be implemented with a device such as the Analog Devices AD538 400 KHz integrated circuit.

Digital Phase Estimation:

A digital embodiment of the phase estimator 156, based on Least Squares using signal digitization, works to estimate the products of the signal magnitude and the cos φ and sin φ factors as described below.

Model the sensor signal as x=a sin(ωt+φ) and decompose it into

$$\begin{matrix}x = a sin ( ω t + φ ) \\ = a cos φsin ω t + a sin φcos ω t \\ = [ sin ω t cos ω t ] [ \begin{matrix}a cos φ \\ a sin φ\end{matrix} ] \\ = \underline{f} ( t ) \underline{p}\end{matrix}$$

Form the least square cost function to be minimized with respect to a cos φ and a sin φ based on measurements of x, x*, taken at t₁, . . . , tₖ as

$$J ( \underline{p} ) = {\sum }_{k = 1}^{K} {[ {x}^{*} ( {t}_{k} ) - \underline{f} ( {t}_{k} ) \underline{p} ]}^{2}$$

solve this linear system using

$$\underline{p} = {[ {\sum }_{k = 1}^{K} {\underline{f} ( {t}_{k} )}^{T} \underline{f} ( {t}_{k} ) ]}^{- 1} {\sum }_{k = 1}^{K} {x}^{*} ( {t}_{k} ) {\underline{f}}^{T} ( {t}_{k} )$$

and calculate phase as

$$φ = {tan}^{- 1} ( \frac{a sin φ}{a cos φ} )$$

This digital approach is alternately implemented as a Kalman filter and uses discrete state transition and measurement relationships to sequentially improve estimates of p as:

pₖ₊₁=pₖ

x(tₖ₊₁)=f(tₖ₊₁)pₖ₊₁

resulting in

$${\underline{k}}_{k + 1} = \frac{1}{\underline{f} ( {t}_{k + 1} ) {\underline{P}}_{k / k} {\underline{f}}^{T} ( {t}_{k + 1} ) + {σ}_{measurement}^{2}} {\underline{P}}_{k} {\underline{f}}^{T} ( {t}_{k + 1} )$$

$${\underline{p}}_{k + 1} = {\underline{p}}_{k} + {\underline{k}}_{k + 1} [ {x}^{*} ( {t}_{k + 1} ) - \underline{f} ( {t}_{k + 1} ) {\underline{p}}_{k} ]$$

$${\underline{P}}_{k + 1} = [ \underline{I} - {\underline{k}}_{k + 1} \underline{f} ( {t}_{k + 1} ) ] {\underline{P}}_{k}$$

$$φ ( {t}_{k + 1} ) = {tan}^{- 1} ( \frac{a sin {φ}_{k + 1}}{a cos {φ}_{k + 1}} )$$

The p₀ and its uncertainty covariance, P₀, are initialized according a priori estimates, and σₘₑₐₛᵤᵣₑₘₑₙₜ is the x* measurement standard deviation.

Regardless of the phase estimator 156 technique, the transport delay to and from the sensor, due to the speed of sound and the distance between the transducers and the sensor, is considered. A simple manner to estimate this delay is to use a coarse estimated distances from the transducers to the beam intersection coupled with the speed of sound in the intervening tissue (TBL 1), and wait that time after changing one of the switches 152 and 154 before re-sampling the phase conditions.

Phase Shifting:

The estimate phase shifter 160 must be able to generate a variable phase version of the reference transducer drive signal 110 and, as those skilled in the art can attest, there are many ways to do this. If the ultrasound oscillator 100 works digitally by creating a sinusoid using a sequence of digital steps, shifting the steps creates a phase shifted version. If the ultrasound oscillator is analog, an all-pass filter shifts phase and not amplitude to create a phased shifted version.

Alternately, since sine 104 and cosine 106 versions of the oscillating signal are available for phase estimation 156, a general phase delay function uses the decomposition

sin(ωt+φ)=sin ωt cos φ+cos ωt sin φ

and an analog embodiment 180 of the estimate phase shifter 160 is illustrated in FIG. 14b where the multiplier stages use an analog multiplier component such as the Analog Devices AD835.

In addition to forming the phase shifted slave precursor drive signal 112, the estimate shifter 160 performs the FIG. 12 algorithm and controls switches 152 and 154, and the phase estimator 156 using hardware and software facilities.

Phase Match and Shifter 108 Implemented Using Power Maximum Seeking:

As shown in

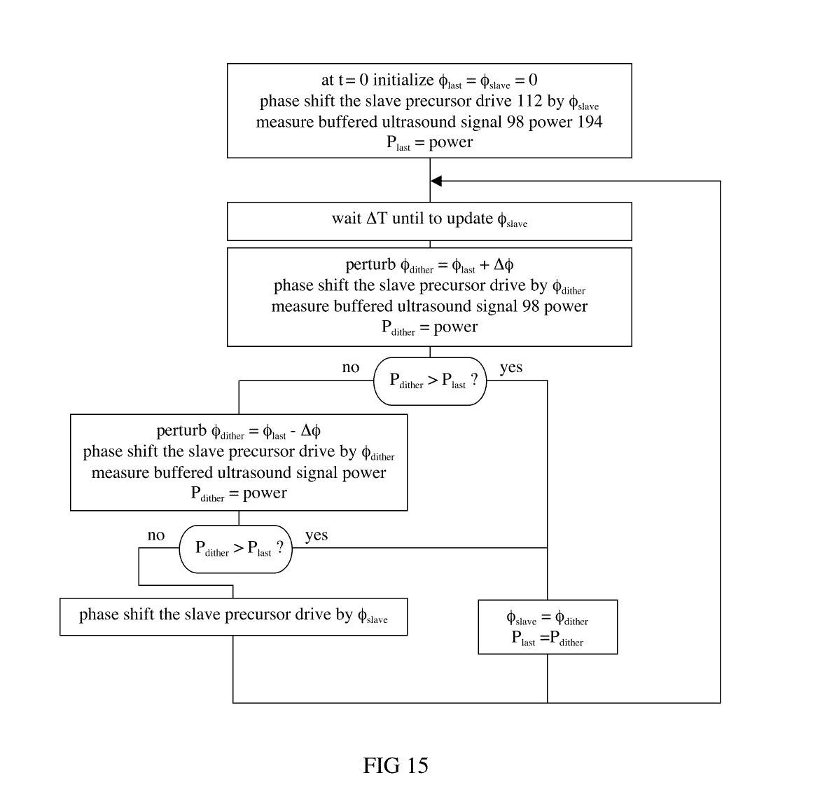

Eq. 5, the optimum φₛₗₐᵥₑ is the value that maximizes power. As shown in FIG. 14 and FIG. 15, a power maximum seeking embodiment 190 of the phase estimator and shifter 108 uses a dither approach to adjust φₛₗₐᵥₑ to maximize the power of the sensor 72 buffered ultrasound signal 98 without switches and without interrupting therapy with switch action. The dither approach, familiar to those skilled in the art, is similar to a gradient maximization scheme where the slave phase φₛₗₐᵥₑ is perturbed a small amount, the resulting power in the reported ultrasound signal is measured, and the phase controlled in the direction of increasing power. A basic algorithm is presented in FIG. 15.

The maximum seeking phase match and shifter 190 perturbs the slave precursor drive signal 112 phase φₛₗₐᵥₑ while the power calculator 192 determines resulting buffered ultrasound signal 98 power 194. As those skilled in the art can attest, power determination has many analog and digital solutions. Since absolute power is not required, only relative power, the preferred approach is to determine the mean value of the square of the incoming signal during a signal acquisition period. The acquisition period is preferably several cycles of the buffered ultrasound signal with, preferably, acquisition starting and stopping at the same position in the cycle (e.g. a positive going zero crossing). The phase perturbation Δφ is on the order of 5°. Not shown in FIG. 15, but understood from discussion above, the transducer to sensor transport delay must be dealt with by waiting the estimated period after changing φₛₗₐᵥₑ and before power is determined.

Intersecting Transducers Using Swept Phase Approach

Whereas the intersecting transducers using match phase approach 50 maximizes peak delivered power by establishing stationary interference patterns with maxima in the beam intersection 78, the intersecting transducers using swept phase 200 avoids stationary interference patterns to mitigate wave cancellation, as seen in FIG. 8. Like the phase matching mode, swept phase provides a power gain of 4× relative to tissue in the non-intersecting beams. Any of the three intersections of FIG. 3b-d apply, and swept phase does not require an ultrasonic sensor, although an optional temperature probe to monitor therapy progress, as per FIG. 1, is considered. Preferably, the Fresnel-Fresnel interference pattern of FIG. 3c is used where therapy is performed in the cylindrical near field and the drive signal frequency f and transducer diameter D define the depth of therapy and divergence angle β used to dissipate ultrasound intensity beyond the target tissue. The drive signal frequency f and the diameter D of the transducer are used, as illustrated in TBL 2, TBL 3 and FIG. 5, to define the divergence angle of the beams to controlling the rate of intensity dispersion, and the depth of therapy.

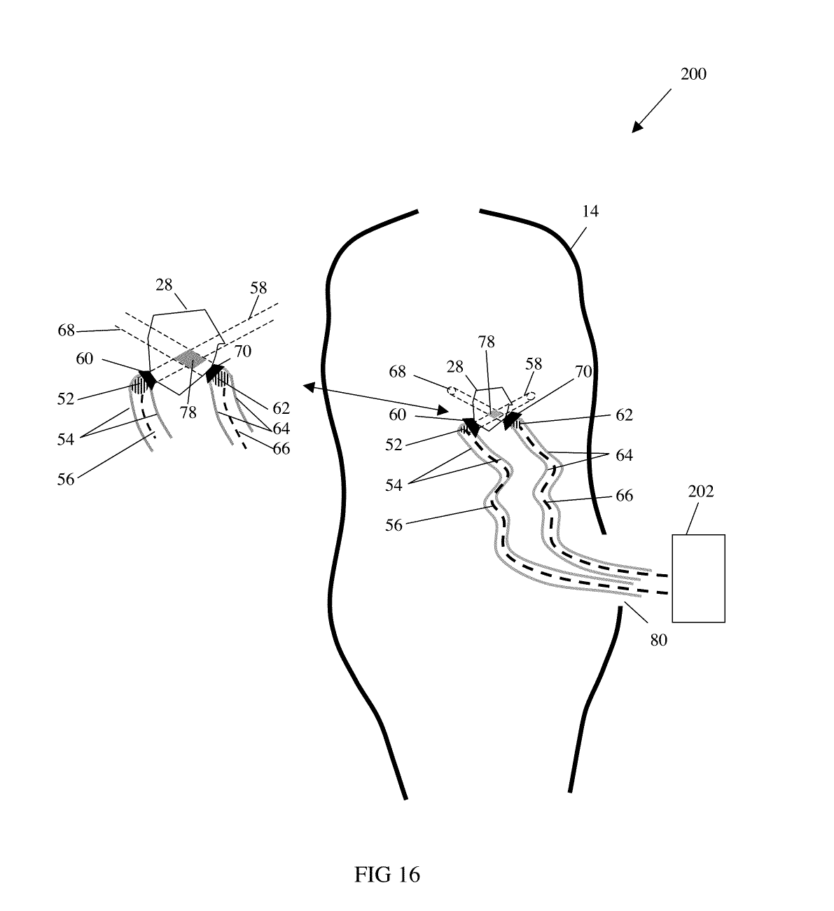

As illustrated in FIG. 16, except for not requiring sensor 72 and using phase sweeping driver generator 202 rather than a phase matching driver generator 82, the swept phase approach 200 is similar in description to the phase matching approach 50 of FIG. 9.

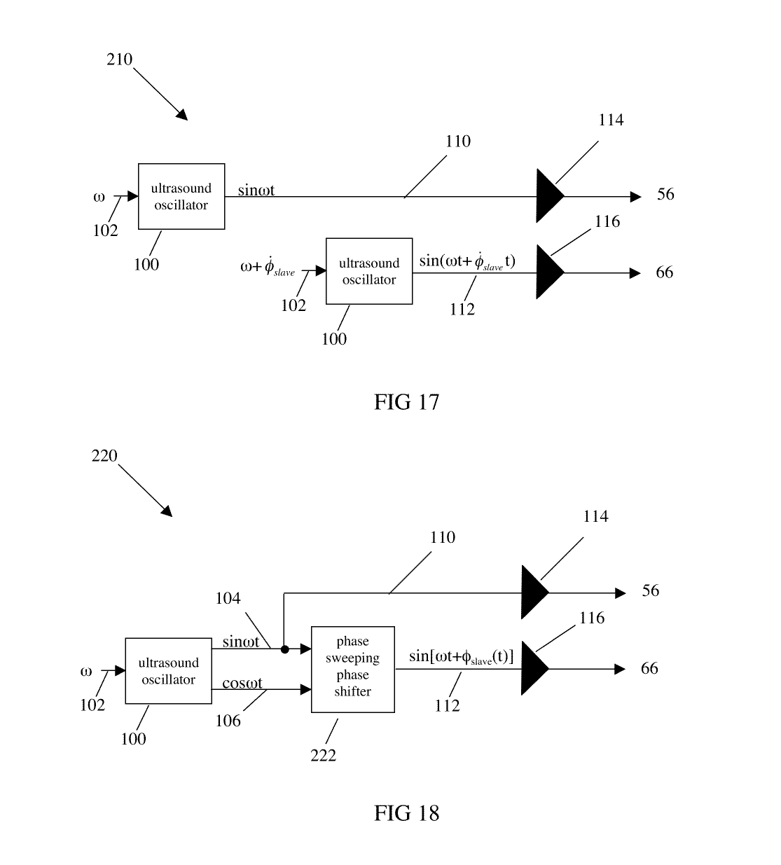

FIG. 17 presents an embodiment 210 of the phase sweeping drive generator 202 using two ultrasound oscillators 100. Based on Eq. 3, the first ultrasound oscillator 100, operating at frequency 102 ω, generates sin ωt that is the precursor reference drive signal 110, and the second ultrasound oscillator, operating at a slightly different frequency 102 ω+φₛₗₐᵥₑ, generates the sin(ωt+φₛₗₐᵥₑt) phase swept slave precursor drive 112. If φₛₗₐᵥₑ is a constant, a linearly swept phase is generated, and φₛₗₐᵥₑ can be varied for other affects. The accuracy of this embodiment is limited by the frequency accuracy of the two ultrasound oscillators.

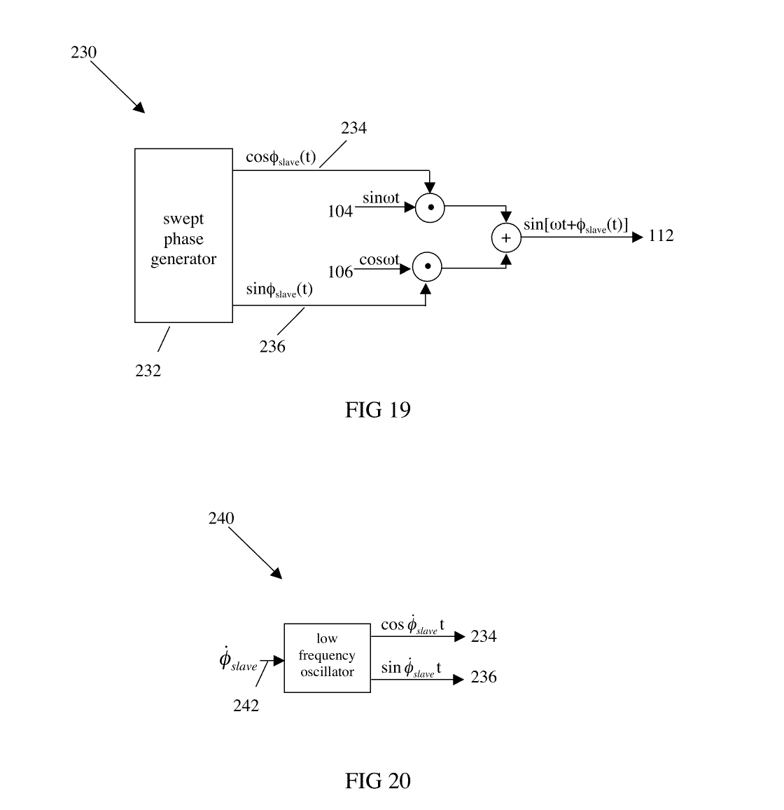

FIG. 18 illustrates the preferred embodiment 220 of the phase sweeping drive generator 202 using a single ultrasound oscillator 100 to generate the reference precursor drive signal 110 and a phase sweeping phase shifter 222 to generate the slave precursor drive signal 112. As illustrated in FIG. 19, and based on the identity

sin([ωt+φₛₗₐᵥₑ(t)]=sin ωt cos [φₛₗₐᵥₑ(t)]+cos ωt sin [φₛₗₐᵥₑ(t)]

an embodiment 230 of the phase sweeping phase shifter 222 uses a swept phase generator 232 to generate the sine 236 and cosine 234 functions of the time varying φₛₗₐᵥₑ. The cosine and sine values are provided by the swept phase generator 232.

The φₛₗₐᵥₑ(t) sweep rate is on the order of a few hundred Hz, and FIG. 20 illustrates an embodiment 240 of the swept phase generator 232 using a low frequency oscillator 244 that linearly sweeps phase at rate φₛₗₐᵥₑ as per Eq. 3. Using frequency 242 φₛₗₐᵥₑ, the low frequency oscillator produces the sine 236 and cosine 234 values of the time varying phase φₛₗₐᵥₑ(t)=φₛₗₐᵥₑ t. If φₛₗₐᵥₑ is a constant, a linearly swept phase is generated, and φₛₗₐᵥₑ can be varied for other affects.

### Satisfying the Objects of the Invention

The object of the invention was earlier state to provide a therapeutically effective ultrasound device that can go where HIFU cannot, is safer, less expensive, does not require an MRI, and handles larger incremental volumes. These objects are satisfied: the multi-beam ultrasound device:
is small and used within the body in the near vicinity of the target tissue, and can be inserted using minimally invasive methods;
is safer because is in the near vicinity of the target tissue and under manual control by the medical professional who can see bones or gas or other scattering obstructions;
conventional ultrasound transducer are used and does not require the complexity, expense, and size of the HIFU emitters and drive electronics;
an MRI is not required as the device is in the hands of the medical practitioner who can visibly guide its beams; and
larger incremental volumes are handled, the intersection of beams rather than the focus of a cone.

### OTHER EMBODIMENTS

While several illustrative embodiments of the invention have been shown and described, numerous variations and alternate embodiments will occur to those skilled in the art. For example:
including more than two slave transducers;
reference and slave transducers arranged external to the patient surface;
the sensor incorporated within the reference or slave transducer;
using focused rather than unfocused transducers;
in addition to therapeutic applications, include industrial uses; and
selecting the transducer drive frequency and geometry in response to the desired depth of therapy as per FIG. 5.

Such variations and alternate embodiments, as well as others, are contemplated and can be made without departing from the spirit and scope of the invention as defined in the appended claims.

What is claimed is:

1. An ultrasound device, comprising a plurality of acoustic transducers forming a plurality of acoustic pressure beams, where said beams are arranged to intersect and interfere and where their relative phases are controlled to enhance interference in a manner that increases the device interaction with the physical state of material at the intersection.

2. The ultrasound device of claim 1 including a sensor located at the intersection, said sensor having an output signal responsive to the acoustic pressure and where said relative phase control is in response to said signal.

3. The responsiveness to said signal of claim 2 includes estimating the phases of the plurality of beams at their intersection and adapting said relative phase control to make said phases equal.

4. The responsiveness to said signal of claim 2 includes estimating the power delivered by the plurality of beams at their intersection and where said relative phase control is adapted to maximize said power.

5. The sensor of claim 2 is further responsive to the temperature at the intersection where said temperature is used to monitor said interaction with said physical state.

6. The sensor of claim 2 where said acoustic pressure responsive sensor is one of said plurality of acoustic transducers.

7. The ultrasound device of claim 1 where said relative phase control is by phase sweeping.

8. The ultrasound device of claim 7 where the plurality of acoustic transducers are driven by a distinct drive signals having distinct frequencies, and phase sweeping is by use of said different frequencies.

9. The ultrasound device of claim 1 where said transducer acoustic pressure beams are focused.

10. The ultrasound device of claim 1 where said intersection lies in the Fresnel-Fresnel zone, where said transducers are responsive to drive signals having a frequencies of operation, and where the effective depth of said interaction by the plurality of beams is selected by said frequencies and the geometries of said transducers in a manner to disperse their beam interaction beyond the intersection.

11. A method to increase the interaction of an ultrasound device, having a plurality of acoustic transducers forming a plurality of acoustic beams arranged to intersect and interfere, with material at the intersection comprising the step of controlling the relative phases of the beams to enhance said interference.

12. The method of claim 11 further comprising the steps
including a sensor located at the intersection, said sensor having an output signal responsive to the acoustic pressure; and
controlling the relative phase in response to said signal.

13. The method of claim 12 where the responsiveness to said signal includes estimation of the phases of the plurality of beams at their intersection and where said relative phase control strives to make said phases equal.

14. The method of claim 12 where the responsiveness to said signal includes estimating the power delivered by the plurality of beams at their intersection and where said relative phase control is adapted to maximize said power.

15. The method of claim 12 further including the step
adapting said sensor to further be responsive to the temperature at the intersection; and
using said temperature is to monitor said interaction with said physical state.

16. The method of claim 12 where said acoustic pressure responsive sensor is one of said plurality of acoustic transducers.

17. The method of claim 11 where said relative phase control is by phase sweeping.

18. The method of claim 17 further including the step of driving the plurality of acoustic transducers by distinct drive signals having distinct frequencies, and phase sweeping is by use of said different frequencies.

19. The method of claim 11 where said transducer acoustic pressure beams are focused.

20. The method of claim 11 further including the steps
arranging said intersection to occur in the Fresnel-Fresnel zone;
adapting said transducers to be responsive to drive signals having frequencies of operation; and
selecting the effective depth of said interaction by the plurality of beams by adapting said frequencies and the geometries of said transducers to disperse their beam interaction beyond the intersection.
